(19) United States
(12) Patent Application Publication
(10) Pub. No.: US 2020/0023149 A1
(43) Pub. Date: Jan. 23, 2020

(54) PORTABLE REBREATHING SYSTEM WITH PRESSURIZED OXYGEN ENRICHMENT

(71) Applicant: Global Rescue Innovation AB, Bettorpsgatan IO (SE)

(72) Inventors: Ola Telby, Karlstad (SE); Jan Olov Stööd, Hammaro (SE)

(21) Appl. No.: 16/037,653

(22) Filed: Jul. 17, 2018

(51) Int. Cl. A61M 16/00; A61M 16/22; A62B 7/02; A62B 7/10

(52) U.S. Cl. CPC A61M 16/0045; A61M 16/22; A62B 7/02; A61M 16/0816; A61M 2210/0618; A61M 16/0093; A62B 7/10

(57) ABSTRACT

This invention relates to a portable rebreathing system, said portable rebreathing system comprising a breathing mask (4), a carbon dioxide scrubber (3), a counter lung (2), ambient air ports and an oxygen supply port (5), with the breathing mask (4) connectable with a mask connector to a common valve housing (X) containing said carbon dioxide scrubber (3), ambient air port and oxygen supply port, and with the counter lung (2) connectable with a counter lung connector to the common housing.

The main features of the invention are two one way check valves close to breathing mask connects to an exhale passage and an inhale passage respectively in a rebreathing mode. A small common valve housing (X) with control valves open a rebreathing circuit to the counter lung when pressure from an oxygen supply source is applied on the control valves, and when pressure drops open a breathing circuit to ambient air. Supply of fresh oxygen is added into the rebreathing circuit directly upstream of or at the inhale check valve.

This design utilizes the oxygen supply efficiently with minimum oxygen losses.

### FIELD OF THE INVENTION

The present invention relates to a portable rebreathing system with pressurized oxygen enrichment, said portable rebreathing system comprising a breathing mask, a carbon dioxide scrubber, a counter lung and an oxygen supply port connected via a hose to a pressurized oxygen source.

### BACKGROUND INFORMATION

The surrounding air consists of about 21% of oxygen. At each inhalation, the body extract about 5% units of that oxygen and the remaining 16% of oxygen is exhaled to the atmosphere again together with CO₂ which is about 5% of the volume exhaled. To reduce the amount of oxygen gas needed in a breathing equipment, and make it possible to reuse the oxygen exhaled, closed circuit breathing apparatus also called rebreathers are used. In a rebreather, the produced CO₂ is absorbed in a scrubber material, most often calcium hydroxide or soda lime. Rebreathers can also be used to provide high oxygen fractions for medical purposes without wasting a lot of oxygen.

Several prior art systems provide closed re-breathing systems to be used in oxygen depleted or toxic environment. In those system is most often used a carbon dioxide scrubber for the exhalation flow that allows the exhaled air flow to be used again during inhalation. This type of rescue breathing system is typically used for miners or people caught in other areas with toxic fumes.

Some of this type of rescue breathing systems also includes non pressurised oxygen generators that may be activated chemically by mixing chemicals or using a special ignitable oxygen producing candles. With oxygen generators, could the operating time for the rescue breathing systems be extended and a small volume of oxygen is added into the rebreathing circuit keeping the total breathing volume constant. Examples of these re-breathing systems could be seen in;
GB2189152, Emergency escape breathing apparatus, with one-way valves in breathing mask, using counter lung connected to an O₂ tank covering the entire head and a CO₂ scrubbing filter.
GB2233905; Emergency escape breathing apparatus, with one-way valves in breathing mask, using counter lung covering the entire head and a filter capable of both CO₂ scrubbing and O₂ generation.
U.S. Pat. No. 5,113,854, Protective hood with CO₂ scrubbing and a cylinder supplying oxygen into the hood.
US2011/0277768, Protective hood with valves preventing inhalation via scrubber and a cylinder supplying oxygen into the hood.

Still a number of rebreathing systems has been proposed such as
U.S. Pat. No. 4,205,673 (1980), with an ignitable oxygen producing candle;
U.S. Pat. No. 4,172,454 (1979), with a complete protection suit;
U.S. Pat. No. 4,246,229 (1981), with a chemical oxygen generator;
U.S. Pat. No. 4,817,597 (1989), with heat dissipating channel over the counter lung;
U.S. Pat. No. 5,267,558 (1993), Chemical oxygen generator with flow distributor trough scrubber;
US2014/0014098; with visible indicator for oxygen shortage

Re-breathing systems has also been proposed for controlled treatment of persons with reduced lung capacity, or otherwise show low oxygen saturation in the blood. In such cases is also an increased oxygen content in the inhaled flow sought for, sometimes raised from the normal 21% O₂ content in ambient air and up to 100% O₂ content.

Rescue vehicles are often equipped with large oxygen tanks that may supply pure oxygen into breathing masks or into nozzles applied into the nostrils. The problem is that the oxygen is consumed rapidly and most of it is wasted during exhalation.

Another problem is the total weight of the system which cause strains on the rescue personnel and may prevent quick appliance to patients in real field situations. Conventionally has the oxygen been supplied from a large pressurized oxygen cylinder, in loaded state pressurized to 200-300 bars, directly to a breathing mask covering the mouth and nose, or via nozzles entered directly into the nostrils. However, a huge part of the oxygen supplied has been wasted.

Most of the rebreathing systems developed for rescue purposes in oxygen depleted environment could not be used for intensified oxygen treatment, so rescue personnel need to bring along bulky and heavy oxygen tanks that conventionally could only be connected to one person at the time.

The need for many small rebreathing systems to be used for intensified oxygen treatment became evident in Sweden after a large fire in a discotheque, where almost a hundred youngsters was rescued but with smoke affected lungs. Even if a tenfold of ambulances arrived at the accident scene was only a tenfold of persons given the aid of increased oxygen treatment. This since each rescue vehicle only had one bulky oxygen tank and one connector with a single mouth piece.

WO2014/035330 disclose a rebreathing system used for extending supply of oxygen to the rebreathing circuit. As disclosed in WO2014/035330 is the necessity and use of this rebreathing system in detail described. In this rebreathing system is a single two-way valve used to shut off a breathing passage when the pressure of the external oxygen source drops. However, in the rebreathing mode the dead volume of exhaled air flow that has not passed the CO₂ scrubber, or only partially has passed the CO₂ scrubber, is inhaled in next inhale stroke.

### SUMMARY OF THE INVENTION

The present invention is a further development of WO2014/035330 with improved functionality that minimizes the dead volume of exhaled CO₂ rich air that may be inhaled and minimizes the necessary addition of oxygen from the oxygen source and could establish a higher concentration of oxygen in the inhalation flow at need. This would increase effective operational time for the portable rebreathing equipment and reduce weight and volume of the pressurized oxygen source.

The improved functionality is obtained by arranging two one way check valves close to the breathing mask that opens or close an inhale or an exhale passage, thus arresting the larger part of the exhale flow in the exhalation passage and only allow flow of air that has passed the scrubber to be guided to the user during inhalation.

It is a primary object of the present invention to obtain a rebreathing system that may be used in both rescue situations (in oxygen depleted or toxic environment) as well as capable to be used on persons suffering decreased lung capacity or low oxygenation of the blood. Hence, the rebreathing system must be able to supply a normal oxygen content (about 21%) and an elevated oxygen content up to over 90%.

Another primary objective is to obtain a rebreathing system that utilize the source of oxygen at the most, enabling as long operating time of the rebreathing system as possible.

Yet another objective is that the rebreathing system must be small and with low weight, so that several units may be brought to an accident scene in an ordinary rescue vehicle, and usage of these systems should not bring about muscle strain or injuries to the rescue personnel.

According to a preferred concept is the invention related to a portable rebreathing system for enriched oxygen breathing, closed rebreathing at nominal oxygen concentration as well as adjustable oxygen enrichment of breathing ambient air. This multi task functionality is of outmost importance for rescue personnel being sent out to accidents where it is unknown what kind of situations will be discovered at the scene of the accident. Said portable rebreathing system comprising;
a breathing mask;
a common valve housing connected with a mask connector to the breathing mask;
a carbon dioxide scrubber connected with a scrubber connector to the common valve housing;
a counter lung connected with a counter lung connector to the carbon dioxide scrubber;
an oxygen supply port and at least one ambient air port arranged in the common valve housing;

and a pressurized oxygen source connected to the oxygen supply port via a hose. These parts of the rebreathing system are essential parts for enablement of the multi task capability of the rebreathing system. In order to obtain the improved usage of oxygen efficiency and thus extended treatment time is the rebreathing system characterized in that

the mask connector connects to a primary chamber in the common valve housing with a total dead volume of less than 10 centiliters, said primary chamber in turn connected to a first exhale passage containing said carbon dioxide scrubber via a first one way check valve and a second inhale passage containing said counter lung via a second one way check valve. This configuration reduces the volume of oxygen depleted exhalation air that may be inhaled in next inhalation phase. Moreover, the rebreathing system also includes

an ambient control valve in the common valve housing connected to a first ambient air port, regulating flow to and from the first ambient air port to said inhale passage. The ambient control valve enable adjustment of the amount of ambient air mixed into the inhalation flow. Moreover, the rebreathing system also includes

a rebreathe control valve in the common valve housing opening an alternative breathing passage connected to a second ambient air port when no oxygen pressure is applied in the oxygen supply port. The pressure responsive rebreathe control valve automatically shifts to rebreathing mode and guarantees that during oxygen enrichment will no fresh oxygen be wasted to ambient and instead caught in the counter lung for reuse. Moreover, said rebreathing control valve closing the alternative breathing passage connected to the second ambient air port when oxygen pressure is applied in the oxygen supply port, thus opening a rebreathe passage comprising the first exhale passage via the first one way check valve and a second inhale passage via a second one way check valve.

This basic set up with two one way check valves close to breathing mask, and a common valve housing with an ambient cvontrol valve and a rebreathe control valve are the basic components of the invention.

According to a first embodiment of the invention may also the portable rebreathing system include that said rebreathe control valve is arranged in parallel with the first one way check valve and the second inhale passage all valves facing the primary chamber in the common valve housing. This location of the rebreathe control valve close to the breathing mask allow short breathing channels to ambient when no oxygen pressure is applied, and hence a low flow restriction for breathing.

In a preferred embodiment of the inventive portable rebreathing system is the pressurized oxygen supply added to the inhale passage in a mixing valve located immediately upstream of the second one way check valve, thereby adding the fresh oxygen close to breathing mask and using the mixing effect of the mixing valve and the turbulence from the one way check valve for a thorough mixing of oxygen into the inhalation flow. For controlled addition and mixing of oxygen into the inhalation flow could preferably a mixing valve in form of a Venturi nozzle be used. Adding oxygen into the flow restriction of the Venturi nozzle, trough which the entire inhalation flow passes, support oxygen flow as the inhalation flow in the Venturi increase speed of flow and thus a drop of pressure at the point of where oxygen is to be added.

In order to control consumption of valuable oxygen is preferably the mixing valve equipped with at least one constant flow rate nozzle for supply of the pressurized oxygen at a rate of 0.5-1.5 liter of oxygen per minute. Such a minimum order of oxygen supply covers most typical applications and would safeguard that the portable rebreathing system never may establish critical low oxygen content. As mentioned is at least one constant flow rate nozzle used, but several additional oxygen nozzles may be used. The number of nozzles may increase in proportion to volume of air passing, i.e. if the person to be treated is hyperventilating or is exposed to physical exercise, more oxygen may be added.

In one embodiment said rebreathe control valve may have a disc like closing member movable between a retracted position opening the alternative breathing passage and an extended position closing the alternative breathing passage, and wherein the disc like closing member is an end wall of a pressure chamber connected to the oxygen supply port. Such simple closure mechanism may comprise a pressure chamber in form of a flexible bellow or a hollow piston, that move the disk like closing member.

In a preferred embodiment is the ambient control valve in the common valve housing a rotatable disc with a first passage in register with the first ambient air port (7a) and a second passage in register with the counter lung, said first and second passages showing a degree of restriction being conversely to each other and dependent on the rotational position of the ambient control valve. By rotating the ambient control valve could the relative proportion of ambient air to the inhalation flow be regulated seamlessly from 0% and up to 100%, Said regulation preferably made by a manual control member accessible on the outside of the common valve housing, and connected with the ambient control valve.

Alternatively to this strict manual control of the ambient control valve could this valve be controlled in part by applied oxygen pressure. The ambient control valve in the common valve housing may be an axially movable valve body keeping the connection to an ambient air port open by spring means and when oxygen pressure is applied on the opposite side of the movable valve body closes the connection to the ambient air port, regulating flow to and from the ambient air port to said ibhale passage.

Further, any pressure responsive rebreathe control valve and/or ambient control valve may also be complemented with a pressure control valve included in the common valve housing that manually connects or disconnects the pressure from the oxygen supply port on the rebreathing control valve and/or the ambient control valve. This will allow the rescue personnel to control the mode of the portable rebreathing system.

In yet a preferred embodiment of the inventive portable rebreathing system is the common valve housing containing all control valves and said common valve housing had overall dimensions nor exceeding 150×100×100 millimeters. This compact design enable a rescue vehicle to bring along a large number of portable rebreathing systems, that may be used for a corresponding number of victims, offering all kind of oxygen treatments to victims suffering from reduced oxygen saturation problems to escape breathing equipment's needed when being evacuated trough toxic or oxygen depleted environment.

In yet a preferred embodiment of the inventive portable rebreathing system is the common housing containing a manually adjustable control member capable of adjusting the position of the ambient control valve connected to the ambient air port, opening flow to and from the ambient air port to said inhale passage dependent on the position of the manually adjustable control member. By this manual control could the rescue personnel adjust the order of oxygen enrichment that may be needed. By gradual closing of this valve may the oxygen concentration increase, which may be needed if the victim is showing low oxygen saturation in the blood due to affected lungs or other causes.

In a further preferred embodiment of the inventive portable rebreathing system is the position of the rebreathe control valve depending on the oxygen pressure established in the oxygen supply port applied at one side of the rebreathe control valve body and a counter force from a return spring applied on an opposite side of the rebreathe control valve body. Hence, the position of the rebreathe control valve could be closed by the return spring and when the oxygen pressure is applied will the rebreathe control valve open a rebreathe passage.

In a further preferred embodiment of the inventive portable rebreathing system is the carbon dioxide scrubber connected with a detachable scrubber connector to the common valve housing in one end of the carbon dioxide scrubber and in the other end is the carbon dioxide scrubber connected with a detachable counter lung connector to the counter lung forming a part of the exhale passage from the rebreathe control valve via said carbon dioxide scrubber and finally to the counter lung, said carbon dioxide scrubber also including a by-passing channel connecting the counter lung to the inhale passage.

According to an additional preferred embodiment of the inventive portable rebreathing system are also the active chemicals of the carbon dioxide scrubber contained in a detachable cassette insert able into the housing of the carbon dioxide scrubber or detachable from the common valve housing.

### BRIEF DESCRIPTION OF THE DRAWINGS

The foregoing aspects and advantages of this invention will become more readily appreciated as the same become better understood by reference to the following detailed description, when taken in conjunction with the accompanying schematically drawings, wherein:

FIG. 1a shows a side view in a cross section of a first schematic embodiment of the rebreathing system according to the invention, here in the basic set up in ambient mode;

FIG. 1b shows same side view as in FIG. 1a but here in the oxygen enrichment mode;

FIG. 1c, shows same side view as in FIG. 1a but here in the full rebreathing mode;

FIG. 1d, shows the disc like control valve in a first rotational position;

FIG. 1e, shows the position of a control knob switchable between an ambient and Oxygen enrichment position;

FIG. 2a shows a side view in a cross section of a second schematic embodiment of the rebreathing system according to the invention, here in the basic set up in ambient mode;

FIG. 2b shows same side view as in FIG. 2a but here in the oxygen enrichment mode;

FIG. 2c, shows same side view as in FIG. 2a but here in the full rebreathing mode;

FIG. 2d, shows same side view as in FIG. 2a but here in the reduced rebreathing mode;

FIG. 2e, shows same side view as in FIG. 2a but here with all additional parts disconnected from the common valve housing;

FIGS. 3a and 3b shows the position of a control knob, used in the second embodiment of the rebreathing system, switchable between an ambient and rebreathe position respectively;

FIG. 4 shows the position of a control knob, used in the second embodiment of the rebreathing system, adjustable between minimum and maximum ambient flow;

FIG. 5a-5b shows a schematic visualization of a third alternative embodiment of above FIGS. 1a-1b;

FIG. 6a shows an embodiment of the common valve housing of the rebreathing system according to the invention;

FIG. 6b shows schematically the design modules of the common valve housing of the rebreathing system according to FIG. 6a;

FIG.7 shows an embodiment the invention in a small rescue bag with all necessary components;

FIG. 8 shows the difference between conventional oxygen tank systems and the invention in several important aspects; and

FIG. 9 shows one example of one way check valves that may be used.

However, it should be stressed that the drawings only visualize the concepts of the invention, as presentable in 2 dimensional drawings. Some channels may for instance utilize the option to be routed not only in the 2 dimensions shown, but also may be routed in 3 dimensions fully utilizing the total volume of the common valve housing.

### DETAILED DESCRIPTION OF THE ENABLING EMBODIMENTS

The following detailed description, and the examples contained therein, are provided for describing and illustrating the principles of three different enabling embodiments of the invention and are not intended to limit the scope of the invention in any way.

Valve members are using standard symbols for indicating open or closed status, i.e. [symbol] for a closed valve and [symbol] for an open valve.

### First Embodiment

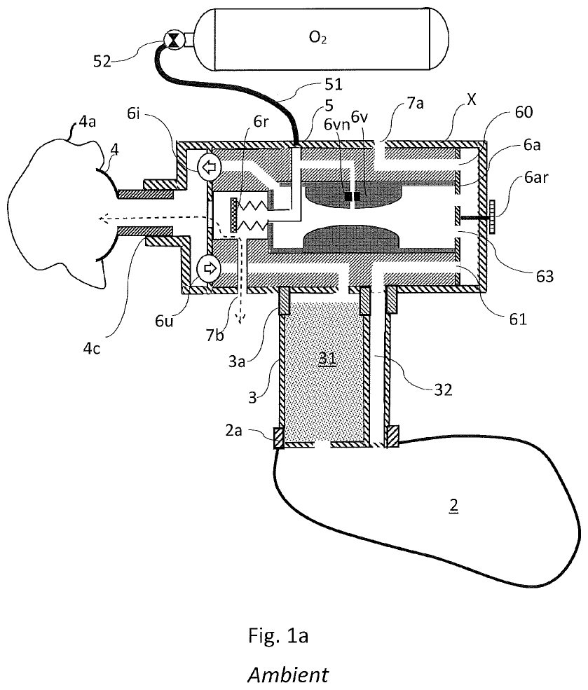

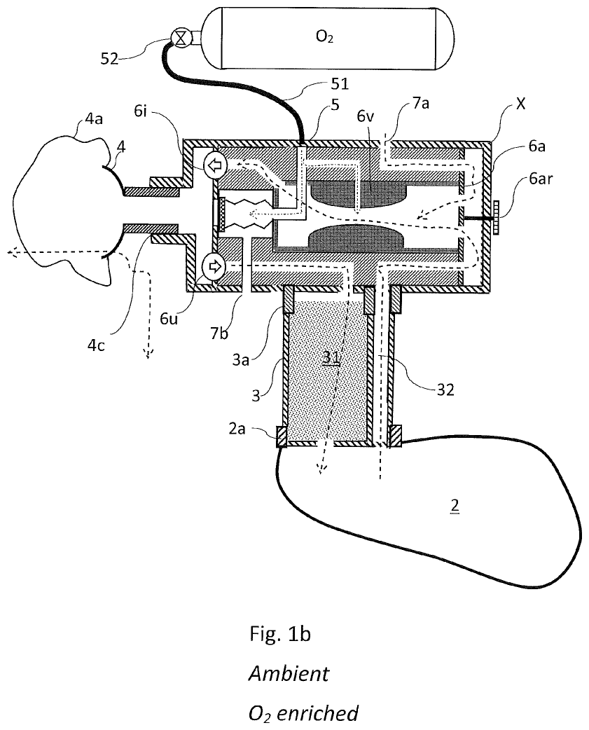

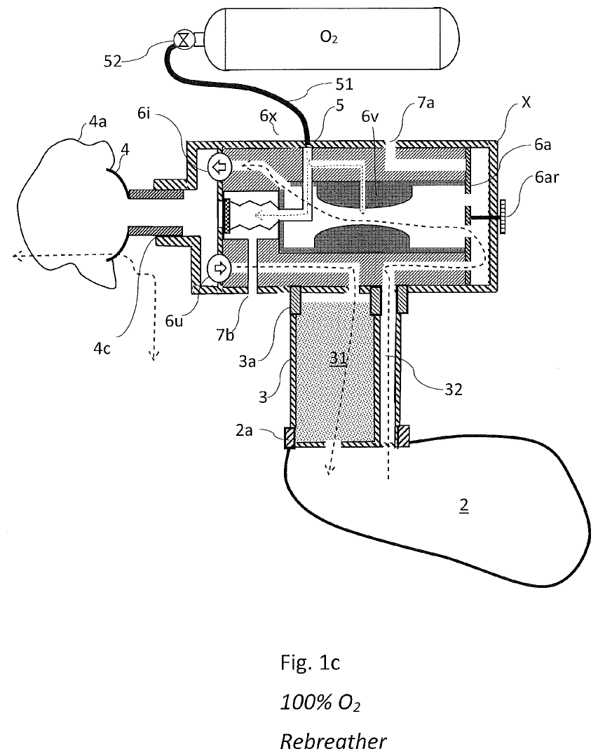

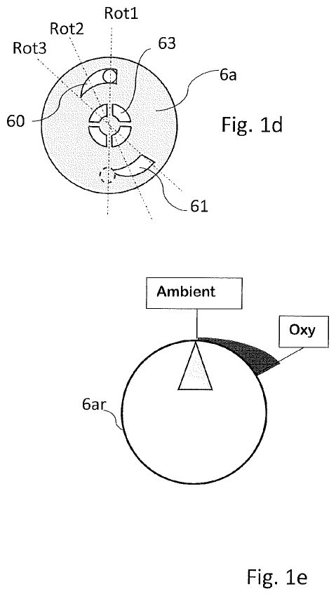

FIG. 1a-d show a first enabling embodiment of the invention in the same cross section side view, but in 3 different operating modes, and FIG. 1e show the same cross section view of this embodiment with all main parts disconnected from each other. The 3 different operating modes are hereafter called;
1) “Ambient”, shown in FIG. 1a;
2) “Ambient O₂ enriched”, shown in FIG. 1b;
3) “100% O₂ Rebreather”, shown in FIG. 1c.

First embodiment, “Ambient” mode, FIG. 1a:

In this mode, the portable rebreathing system is simply allowing breathing towards ambient air, and used when mounting the breathing mask 4 over nose and mouth of a person to be treated. The breathing mask is kept in place by an adjustable flexible neck strap 4a and connects via a mask connector 4c to a common valve housing X. The mask connector 4c connects to a primary chamber in the common valve housing X with a total dead volume of less than 10 centiliters in the primary chamber, said primary chamber in turn connected to a first exhale passage via a first one way check valve 6u and a second inhale passage via a second one way check valve 6i.

As seen here is a combined inhale and exhale passage ending/starting at the open ambient air port 7b located on the lower part of the common housing X.

A pressurized oxygen source O₂ is connected via a closed valve 52 to an oxygen supply port 5 in the common valve housing X. When the valve 52 is opened is the supply port fed with oxygen at a predetermined overpressure in relation to ambient pressure, which overpressure is set by a conventional pressure reduction valve (not shown per se) in the valve 52. Typically, the pressure in the oxygen source may be 200-300 bar, and the pressure applied on the oxygen supply port may be 0.5-1 bar above ambient pressure.

The common valve housing X contains all control valves for adjusting the rebreathing system from simple ambient mode shown in FIG. 1a, to the 2 additional modes, i.e. ambient mode with O₂ enrichment, or 100% O₂ rebreather to be used when treating people showing severe symptoms of low oxygen saturation in the blood or 20-100% O₂ rebreather to be used when treating people showing less degree of symptoms of low oxygen saturation in the blood. In the 100% O₂ rebreather mode could the equipment be used as rescue equipment when evacuating people from and trough toxic environment.

The common valve housing contains an ambient control valve 6a that in ambient mode is fully open towards the upper ambient air port 7a. As shown here is this ambient control valve 6a disc rotated towards any selected position. Further a rebreathe control valve 6r is fully open towards the lower ambient air port 7 as the disc 6r is retracted to the fully open position by the bellow not being pressurized, as no pressure from the oxygen source is applied is led to the bellow.

First Embodiment, “Ambient O₂ Enriched” Mode, FIG. 1b:

In this mode of operation is the portable rebreathing system used in nontoxic environment when the person to be treated is to be supplied with breathing air enriched with oxygen.

A pressurized oxygen source O₂ is connected via an open valve 52 to an oxygen supply port 5 in the common valve housing X. A flow channel in the common valve housing X is connected between the oxygen supply port 5 and a mixing valve 6v located immediately upstream of the second one way valve 6i in the inhale passage.

Further, the bellow for the closure disc in the rebreathe valve 6r is also pressurized when the valve 52 is open, and effectively closes the connection to the ambient port 7a. Breathing must now take place through the two one way valves 61 and 6u.

The mixing valve 6i is in this embodiment a Venturi nozzle that results in a reduction of pressure of the inhalation flow as it passes the Venturi constriction, and the pressurized oxygen supply duct is connected to this restriction. Thus, the pressurized oxygen is added into the inhalation flow kept at high flow rate and low pressure. The Venturi nozzle 6v is also preferably located close, i.e. within a distance shorter than 2 cm, to the second one way valve 6i that by effect also adds an additional mixing effect as an adjustable restriction in the inhalation flow passage. The added oxygen will thus be added into the inhalation flow close to the mouth piece, and effectively mixed into the inhalation flow.

The ambient control valve 6a is rotated into a position by the manual control member 6ar to a non-restricting position towards the upper ambient port 7a. In this mode is oxygen from the counter lung inhaled and complementary ambient air. As will be described later could the proportions of ambient air be regulated by the selected rotational position of the control valve 6r.

In this mode of operation is the carbon dioxide scrubber used, but the volumes inhaled from the counter lung would lack any CO₂, but also includes all residual oxygen that has been added. Thus no waste of oxygen is at hand.

First Embodiment, “100% O₂ Rebreather” Mode, FIG. 1c:

In this mode of operation is the portable rebreathing system used for highest possible oxygen content in the inhalation flow and without any loss of oxygen from the pressurized oxygen source. Typically, this mode is used for treating a person with problems reaching appropriate oxygen saturation level in the blood. Ambient air with its 20% oxygen level is not used at all for breathing and pure oxygen is added to the inhalation flow in replacement of the carbon dioxide caught in the carbon dioxide scrubber. Over time the oxygen concentration will increase and no losses of valuable pure oxygen is at hand.

A pressurized oxygen source O₂ is connected via an open valve 52 to the oxygen supply port 5 in the common valve housing X. Besides supplying oxygen to the mixing valve 6v will the oxygen pressure also be led to the bellow in the control valve 6r that closes connection to the ambient air port 7b.

As in previous mode the pressurized oxygen is added into the inhale flow kept at high flow rate and low pressure in the Venturi nozzle 6v.

The manual control member 6ar is rotated to a position closing connection with the upper ambient air port 7a.

In this mode of operation is the carbon dioxide scrubber used, as oxygen concentration in the inhalation flow increases after removal of CO₂ and continuous addition of oxygen.

While FIG. 1c show that the connection to the ambient air port 7a is closed, with an effect that oxygen concentration will increase, could the connection to the ambient air port be gradually opened resulting in adjustable oxygen content of the inhalation flow.

First Embodiment, Rotational Rebreathe Control Valve, FIGS. 1d and 1e

The function of the rotational rebreathe control valve 6r is shown in FIG. 1d. The disc shaped valve member 6a is equipped with slots 60 and 61 allowing gradual restriction of flow from the counter lung and/or the upper ambient air port 7a. In FIG. 1d is the disc 6a in a first rotational position Rot1 where the connection to the upper ambient port 7a is totally open, but the connection to the counter lung is totally closed. At each inhalation in FIG. 1c will thus ambient air be introduced into a chamber located on the right hand side of the disc 6a. From there the ambient air pass through the center openings 63 and towards the Venturi mixing valve 6v.

If the disc 6a is rotated such that the Rot2 position comes in register with the channel to the upper ambient port 7a will also the connection to the counter lung be gradually open. In this position are both connections partially open in a 50/50 relationship. If the disc 6a is rotated such that the Rot3 position comes in register with the channel to the upper ambient port 7a will the the connection to the ambient port be closed while the connection to the counter lung be fully open.

As shown in FIG. 1e could the manual control member 6ar be adjusted in many positions between these 2 modes, either in a position where only ambient air is inhaled, or if turning the manual control member 6ar to the full Oxygen mode, “Oxy”, where air from the counter lung is inhaled, or any intermediate mixing position.

Second Embodiment

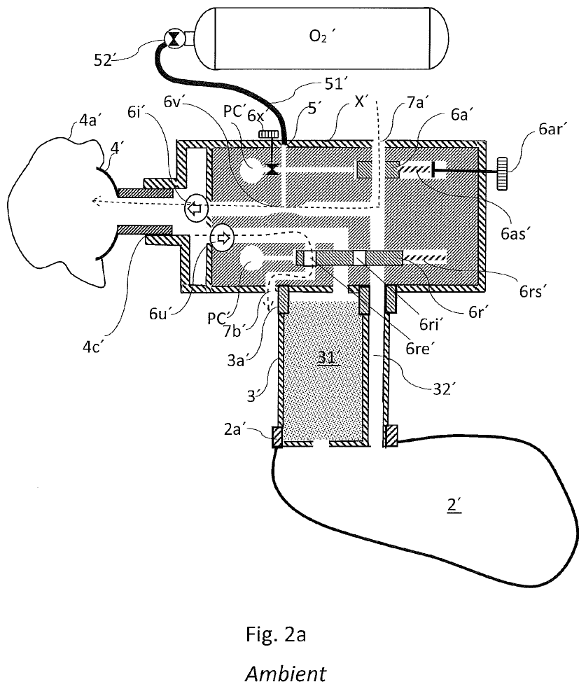

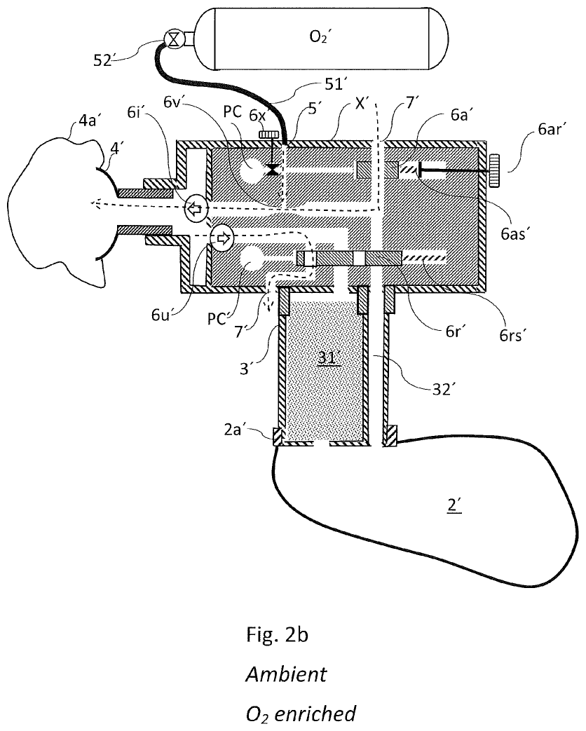

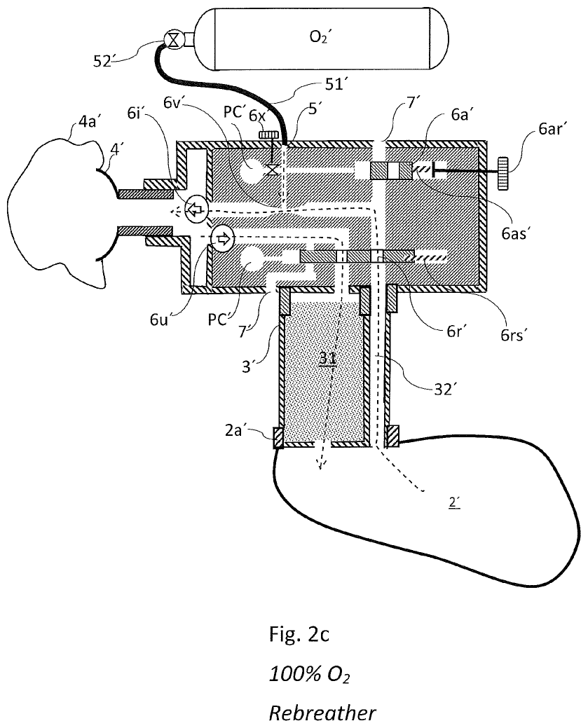

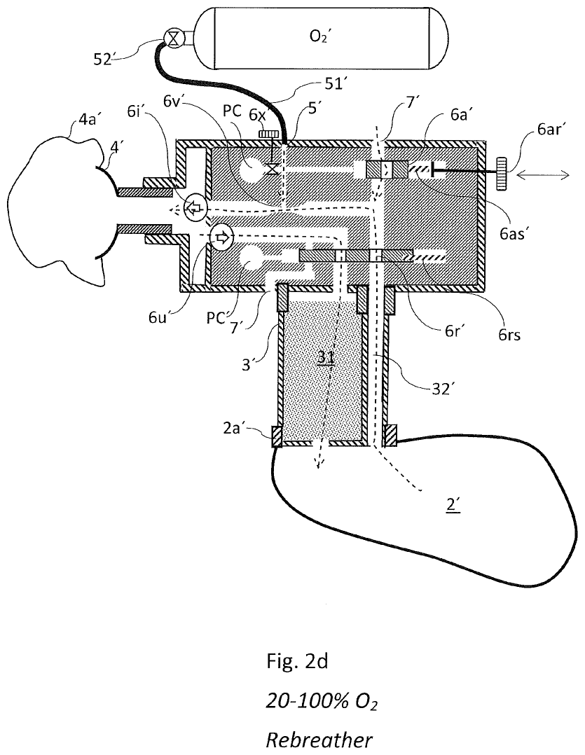

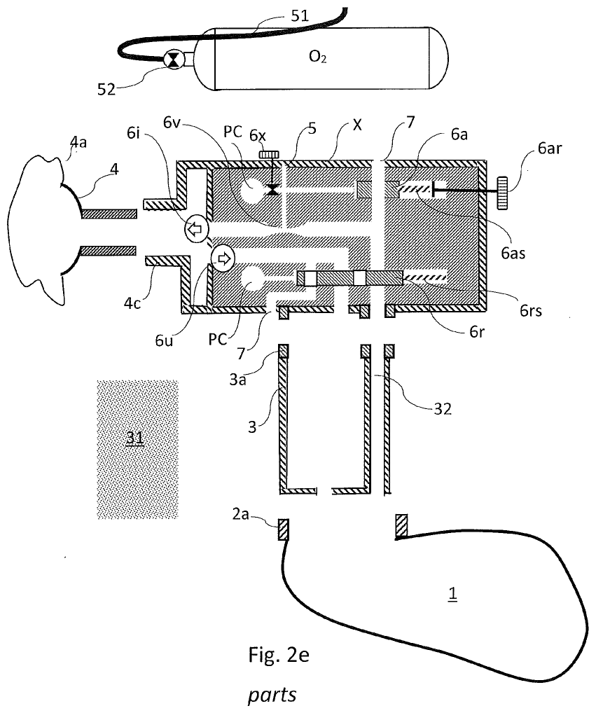

FIG. 2a-d show a second enabling embodiment of the invention in the same cross section side view, but in 4 different operating modes, and FIG. 2e show the same cross section view of this embodiment with all main parts disconnected from each other. The 4 different operating modes are hereafter called;
1) “Ambient”, shown in FIG. 2a;
2) “Ambient O₂ enriched”, shown in FIG. 2b;
3) “100% O₂ Rebreather”, shown in FIG. 2c; and finally
4) “20-100%” Rebreather, shown in FIG. 2d.

Second Embodiment, “Ambient” Mode, FIG. 2a:

In this mode, the portable rebreathing system is simply allowing breathing towards ambient air, and used when mounting the breathing mask 4″ over nose and mouth of a person to be treated. Parts with same function as in the first embodiment will not be described here, and parts with same function is indicated with “′”.

As in the first embodiment, the mask connector 4c ” connects to a primary chamber in the common valve housing X″ with a total dead volume of less than 10 centiliters in the primary chamber, said primary chamber in turn connected to a first exhale passage via a first one way check valve 6u″ and a second inhale passage via a second one way check valve 6i″. As seen here is the exhale passage ending at the open ambient air port 7b′ located on the lower part of the common housing X′. Also, the inhale passage is starting at another open ambient air port 7a′ located on the upper part of the common housing X′.

A pressurized oxygen source O₂′ is connected via a closed valve 52′ to an oxygen supply port 5′ in the common valve housing X′. When the valve 52′ is opened is the supply port fed with oxygen at a predetermined overpressure in relation to ambient pressure, which overpressure is set by a conventional pressure reduction valve (not shown per se) in the valve 52′.

The common valve housing X′ contains all control valves for adjusting the rebreathing system from simple ambient mode shown in FIG. 2a, to the 2 additional modes, i.e. ambient mode with O₂ enrichment, or adjustable rebreathing mode.

The adjustable rebreathing mode set between
either 100% O₂ rebreather to be used when treating people showing severe symptoms of low oxygen saturation in the blood, or
20-100% O₂ rebreather to be used when treating people showing less degree of symptoms of low oxygen saturation in the blood.

In 100% rebreathing mode, could the equipment be used as rescue equipment when evacuating people from and trough toxic environment.

The common valve housing contains an ambient control valve 6a that in ambient mode is fully open towards the upper ambient air port 7. As shown here is this ambient control valve 6a pressed towards the fully open position by a return spring member 6a s as no pressure from the oxygen source is applied on the other end of the control valve 6a. Further a rebreathe control valve 6r is fully open towards the lower ambient air port 7 as a common control valve body 6r is pressed to the fully open position by a return spring member 6r s, as no pressure from the oxygen source is applied on the other end of the common control valve body 6r.

Second Embodiment, “Ambient O₂ Enriched” Mode, FIG. 2b:

In this mode of operation is the portable rebreathing system used in nontoxic environment when the person to be treated is to be supplied with breathing air enriched with oxygen.

A pressurized oxygen source O₂ is connected via an open valve 52 to an oxygen supply port 5 in the common valve housing X. A flow channel in the common valve housing X is connected between the oxygen supply port 5 and a mixing valve 6v located immediately upstream of the second one way valve 6i in the inhale passage.

The mixing valve 6i is in this embodiment a Venturi nozzle that results in a reduction of pressure of the inhalation flow as it passes the Venturi constriction, and the pressurized oxygen supply duct is connected to this restriction. Thus, the pressurized oxygen is added into the inhale flow kept at high flow rate and low pressure. The Venturi nozzle 6v is also preferably located close, i.e. within a distance shorter than 2 cm, to the second one way valve 6i that by effect also adds an additional mixing effect as an adjustable restriction in the inhalation flow passage. The added oxygen will thus be added into the inhalation flow close to the mouth piece, and effectively mixed into the inhalation flow.

The ambient control valve 6a is pushed to the left in FIG. 1b by the manual control member 6ar to a non-restricting position even if the oxygen pressure is applied on the other end of the ambient control valve 6a.

In this mode of operation is not the carbon dioxide scrubber used, as the rebreathing system is used in a nontoxic environment, and hence the carbon dioxide scrubber cassette is not needed to be replaced after usage.

Second Embodiment, “100% O₂ Rebreather” Mode, FIG. 2c:

In this mode of operation is the portable rebreathing system used for highest possible oxygen content in the inhalation flow and without any loss of oxygen from the pressurized oxygen source. Typically, this mode is used for treating a person with problems reaching appropriate oxygen saturation level in the blood. Ambient air with its 20% oxygen level is not used at all for breathing and pure oxygen is added to the inhalation flow in replacement of the carbon dioxide caught in the carbon dioxide scrubber. Over time the oxygen concentration will increase and no losses of valuable pure oxygen is at hand.

A pressurized oxygen source O₂ is connected via an open valve 52 to the oxygen supply port 5 in the common valve housing X. Besides supplying oxygen to the mixing valve 6v will the oxygen pressure also be led to a toroidal pressure chamber PC via an open pressure control valve 6x. The pressure control valve 6x is manually set by a control member in the common valve housing X.

As in previous mode the pressurized oxygen is added into the inhale flow kept at high flow rate and low pressure in the Venturi nozzle 6v.

The manual control member 6ar is retracted to the right in FIG. 1c, allowing the oxygen pressure applied on the other end of the ambient control valve 6a to push the control valve 6a to a closing position, cutting off any connection to ambient air.

In this mode of operation is the carbon dioxide scrubber used, as oxygen concentration in the exhale flow increases, and this oxygen enriched volume is saved in the counter lung.

Second Embodiment, “20-100% O₂ Rebreather” Mode, FIG. 2d:

In this mode of operation is the portable rebreathing system used for adjustable oxygen content in the inhalation flow and without any loss of oxygen from the pressurized oxygen source. Ambient air with its 20% oxygen level is used in proportion to desired oxygen enrichment needed to reach the oxygen saturation in the blood of the person treated. Typically, the addition of ambient air may follow as soon as the person has been treated in the previously described mode “100% O₂ Rebreather mode” and oxygen saturation in the blood has reached over 90%.

The settings of the valves in the common housing are the same as shown in “100% O₂ Rebreather mode”, but an adjustable amount of ambient air can be added into the inhale passage by adjusting the ambient control valve 6a to the left in FIG. 2d by adjusting the manual control member 6ar. Thus, in an adjustable amount of ambient air, with 20% oxygen concentration, is replacing the pure oxygen added in valve 6v in the inhalation flow.

Second Embodiment, Modular Design:

As shown in FIG. 2e is the rebreathing system designed in 6 modules that could be easy replaced. The same modular design applies also for the first embodiment. These modules are;
i. The breathing mask 4 with its adjustable neck strap 4a and connecting sleeve fitting the mask connector 4c in the common valve housing X;
ii. The pressurized oxygen source O₂, preferably a small 200-300 bar tank, with valve 52 and connection hose 51;
iii. The common valve housing X containing all control valves and control members for selecting any of the 4 operational modes;
iv. The Carbon dioxide scrubber housing 3;
v. The carbon dioxide scrubber cassette 31; and
vi. The counter lung 2.

The parts ii and v need to be replace when consumed after usage, and the modular system design enable these to be replaced at low cost.

Further, if the rebreathing system show some malfunction could part iii be replaced and the old one sent to test or service.

Part vi, which may be a simple transparent plastic bag, may also be replaced if ruptured during handling.

The mask connector, counter lung connector and scrubber connector, 4c, 2a and 3a respectively may be of any suitable design enabling release of modules from each other. The hose 51 may be connected by a simple hose nipple on the common housing. In normal usage, would part iv not need to be replaced, but needs to be disconnected if part iii or part v needs to be replaced.

Second Embodiment, Manual Controls:

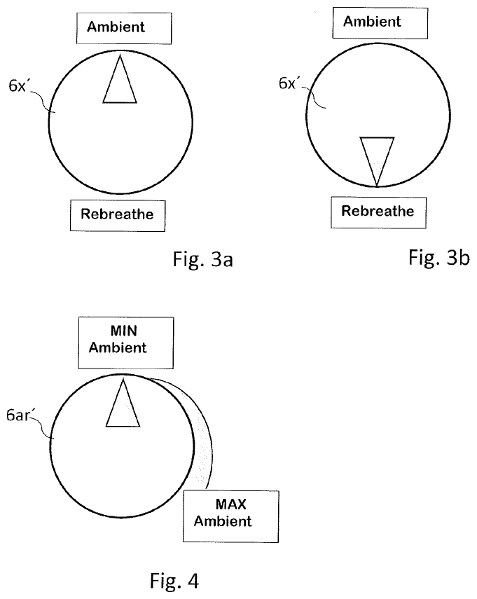

As shown in FIGS. 3a and 3b is the pressure control valve 6x a manual control knob that may be set in either “Ambient” position, shown in FIG. 3a, or in “Rebreathe” position, shown in FIG. 2b. The “Ambient” position is selected when operating in the mode shown in FIGS. 2a and 2b, and the “Rebreathe” position is selected when operating in the modes shown in FIGS. 2c-2d.

As shown in FIG. 4 is the ambient control valve 6ar a manual control knob that may be set in any position between “Min Ambient” and “Max Ambient” position. The “Max Ambient” position is selected when operating in the mode shown in FIGS. 2a and 2b, and the “Min Ambient” position is selected when operating in the mode shown in FIG. 2c, while an adjustable position between “Min Ambient” and “Max Ambient” is selected when operating in the mode shown in FIG. 2d.

### Third Embodiment

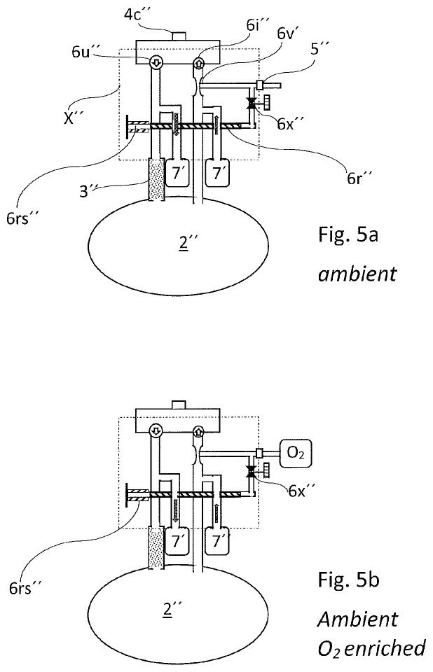

FIGS. 5a-5b show a third enabling embodiment of the invention in the same cross section side view, but in different operating modes, hereafter called;
1) “Ambient”, shown in FIG. 5a;
2) “Ambient O₂ enriched”, shown in FIG. 5b.

Details with similar function as those shown in the first embodiment is given same number but with double apostrophe after the number, i.e. detail 5 become 5″. In this embodiment is a valve body 6r″ used to control opening or closing of ambient airports 7″. The pressure control valve 6x″ is closed in FIGS. 5a and 5b whereby the return spring 6rs″pushes the valve body 6r″ to the right-hand position where the ambient air ports 7″ are fully open.

In the starting position when the pressurized air source is not connected, or connected with valve 52″ closed, inhalation and exhalation passages will be connected to ambient.

When increased oxygen content in inhalation is required is the oxygen source connected to the mixing valve 6v″. (shown in FIG. 5b)

When full rebreathing mode with maximum oxygen concentration is required is the pressure control valve 6x″ opened, and the oxygen pressure will be applied on the right hand side of the valve body 6r″, opening both the exhale passage to the carbon dioxide scrubber and the inhale passage to the counter lung 2″.

When an adjustable amount of oxygen enrichment is required is a manual control member regulated shown in FIGS. 5a-5b. At gradual increase of opening of this valve is the oxygen concentration during inhalation correspondingly decreased, as successively more ambient air, at an oxygen concentration of about 20%, is replacing the pure oxygen. Thus, the oxygen concentration may be adjusted from about 100%, when valve is closed, and down to 20-30% when the valve is fully open.

As could be seen here also in this third embodiment are all control valves and manual control members integrated in the common valve housing X″, which preferably has outer dimensions, W×L, less than 150×100 millimeter.

Overall Design

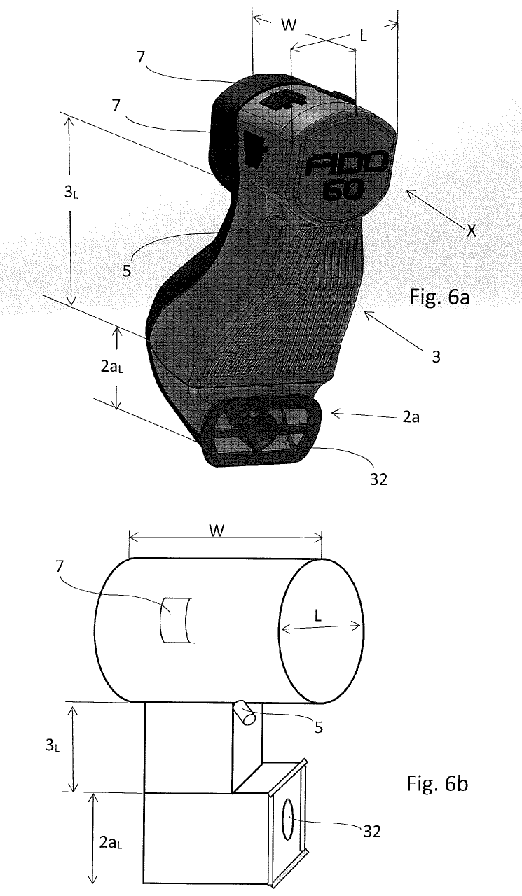

In FIG. 6a is shown a first design prototype, with a common valve housing X with overall dimensions W×L, with ambient air ports 7 and the nipple like oxygen supply port 5. The carbon dioxide scrubber housing 3 is attached to the common valve housing X at an orthogonally direction with an extension 3_{L}. As seen here the outer surface may be provided with profiled surface enhancing the grip during handling. The counter lung connector 2a, connecting the lowermost part of the carbon dioxide scrubber housing 3 with the counter lung, may be provided with a collar for attachment of the inlet of the counter lung, and the by-pass channel 32 is integrated in the center. Exhale flow will thus pass around the by-pass channel 32 and into an attached counter lung, and during inhale will the air pass out from the counter lung to the inlet of the by-pass channel 32. In FIG. 6b are the principle modules 2a, 3, X shown.

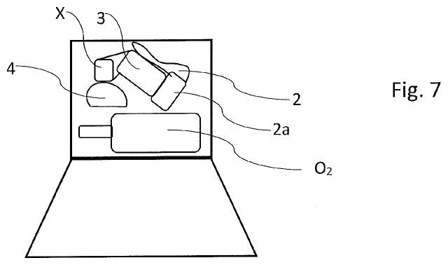

In FIG. 7a is shown a first design prototype how a small box may contain all 6 modules of the rebreathing system, including the
The breathing mask 4 with its adjustable neck strap 4a and connecting sleeve fitting the mask connector 4c in the common valve housing X;
The pressurized oxygen source O2, preferably a small 200-300 bar tank, with valve 52 and connection hose 51;
The common valve housing X containing all control valves and control members for selecting any of the 4 operational modes;
The Carbon dioxide scrubber housing 3;
The carbon dioxide scrubber cassette (mounted inside the housing); and
The foldable counter lung 2.

The principle modules are also shown in FIG. 7b.

Comparison with Conventional Oxygen Treatment Equipment in Rescue Vehicles

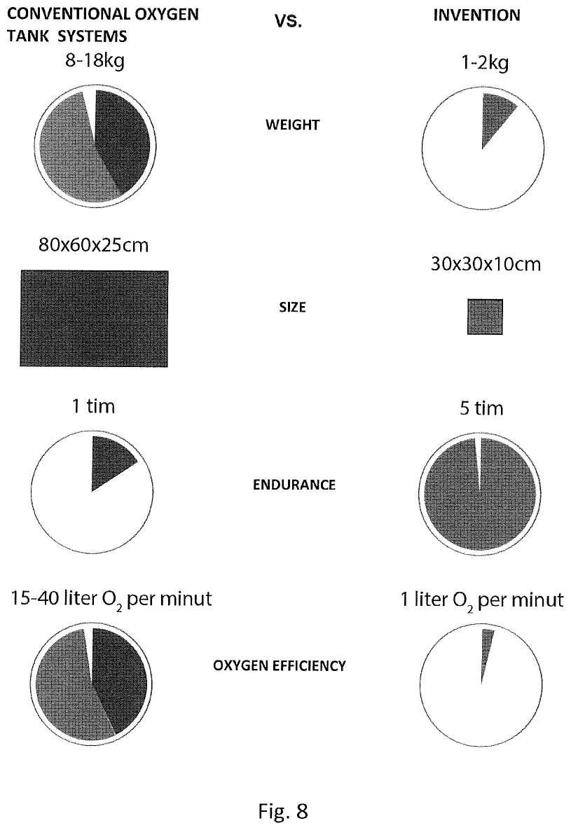

FIG. 8 visualize a comparison between a conventional oxygen treatment equipment that rescue vehicles use, with the invention, comparing
Total weight;
Size;
Endurance;
Oxygen efficiency;

The total weight of the conventional oxygen treatment system is to a large extent dependent of the large pressurized oxygen tank, which typically is a pressure vessel capable of holding 200-300 bar pressure and at a volume of at least 10-12 liter. This volume is needed as the oxygen administration equipment waste a lot of the oxygen supplied.

The overall size of the conventional oxygen treatment system is to a large extent also dependent of the large pressurized oxygen tank, that needs a large part of the limited storage space in the rescue vehicle.

The endurance of the conventional oxygen treatment system is also surprisingly short, typically 1 hour, which is the result of inefficient usage of oxygen. The oxygen efficiency is visualized at the bottom, where the conventional system consumes 15-40 liter per minute, while the invention with counter lung and carbon dioxide scrubber only consumes 1 liter per minute during normal breathing.

Example of One Way Check Valve

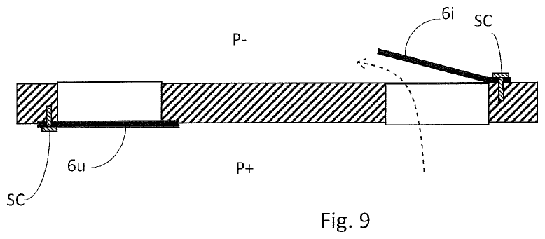

In FIG. 9 is shown one design of two one-way check valves that may be used as the valves 6i and 6u. Here are the valves subjected to under pressure P− in the primary chamber during an inhalation, while a relative overpressure P+ is established on the other side of the valve. The exhalation passage is closed by the valve 6u. The valve flap is preferably a flexible rubber flap that may be kept in place with a screw SC or the like at one point of the periphery. This simple design offers some mixing effect by flow restriction effect from the partially open valve 6i.

Alternatively, the valve flaps could be elastic membranes that have a central fastening member and where flaps open around the entire periphery.

### Alternative Embodiments

The invention is not to be seen as limited by the embodiments described above but can be varied within the scope of the appended claims, as will be readily apparent to the person skilled in the art. For instance, the valves in the housing described above may be of different design and placed in other positions, but with similar functionality as one way check valves, without departing from the inventive concept.

1. A portable rebreathing system for enriched oxygen breathing, closed rebreathing at nominal oxygen concentration as well as adjustable oxygen enrichment of breathing ambient air, said portable rebreathing system comprising
a breathing mask (4),
a common valve housing (X) connected with a mask connector (4c) to the breathing mask (4);
a carbon dioxide scrubber (3/31) connected with a scrubber connector (3a) to the common valve housing (X);
a counter lung (2) connected with a counter lung connector (2a) to the carbon dioxide scrubber (3/31);
an oxygen supply port (5) and at least one ambient air port (7) arranged in the common valve housing;
a pressurized oxygen source (O₂) connected to the oxygen supply port (5) via a hose (51); characterized in that
the mask connector (4c) connects to a primary chamber in the common valve housing (X) with a total dead volume of less than 10 centiliters, said primary chamber in turn connected to a first exhale passage containing said carbon dioxide scrubber (3/31) via a first one way check valve (6u) and a second inhale passage containing said counter lung (2) via a second one way check valve (6i);
an ambient control valve (6a) in the common valve housing (X) connected to a first ambient air port (7a), regulating flow to and from the first ambient air port to said inhale passage;
a rebreathe control valve (6r) in the common valve housing (X) opening an alternative breathing passage connected to a second ambient air port (7b) when no oxygen pressure is applied in the oxygen supply port, and
said rebreathe control valve (6r) closing the alternative breathing passage connected to the second ambient air port (7b) when oxygen pressure is applied in the oxygen supply port; thus opening a rebreathing passage comprising the first exhale passage via the first one way check valve (6u) and a second inhale passage via a second one way check valve (6i).

2. A portable rebreathing system according to claim 1; characterized in that said rebreathe control valve (6r) is arranged in parallel with the first one way check valve (6u) and the second inhale passage all valves facing the primary chamber in the common valve housing (X).

3. A portable rebreathing system according to claim 1; characterized in that the pressurized oxygen supply is added to the inhale passage in a mixing valve (6v) located immediately upstream of the second one way check valve (6i), thereby adding the fresh oxygen close to mouthpiece and using the mixing effect of the mixing valve and the turbulence from the one way check valve for a thorough mixing of oxygen into the inhalation flow.

4. A portable rebreathing system according to claim 3; characterized in that the mixing valve (6v) is a Venturi nozzle.

5. A portable rebreathing system according to claim 3; characterized in that the mixing valve (6v) is equipped with at least one constant flow rate nozzle (6vn) for supply of the pressurized oxygen at a rate of 0.5-1.5 liter of oxygen per minute.

6. A portable rebreathing system according to claim 2; characterized in that said rebreathe control valve (6r) has a disc like closing member movable between a retracted position opening the alternative breathing passage and an extended position closing the alternative breathing passage, and wherein the disc like closing member is an end wall of a pressure chamber connected to the oxygen supply port (5).

7. A portable rebreathing system according to claim 1; characterized in that the ambient control valve (6a) in the common valve housing (X) is rotatable disc with a first passage (60) in register with the first ambient air port (7a) and a second passage (61) in register with the counter lung, said first and second passages showing a degree of restriction being conversely to eachother and dependent on the rotational position (Rot1, Rot2, Rot3) of the ambient control valve (6a).

8. A portable rebreathing system according to claim 7; characterized in that the ambient control valve (6a) is connected to a manual control member (6ar) accessible on the outside of the common valve housing (X).

9. A portable rebreathing system according to claim 1; characterized in that the ambient control valve (6a) in the common valve housing (X) is an axially movable valve body keeping the connection to an ambient air port (7a) open by spring means (6as) and when oxygen pressure is applied on the opposite side of the movable valve body closes the connection to the ambient air port (7a), regulating flow to and from the ambient air port to said inhale passage.

10. A portable rebreathing system according to claim 9 characterized in that a pressure control valve (6x) is included in the common valve housing (X) that manually connects or disconnects the pressure from the oxygen supply port on the rebreathe control valve (6r).

11. A portable rebreathing system according to claim 1 where said common housing (X) contains all control valves (6a, 6r, 6i, 6u) and said common housing has overall dimensions not exceeding (W×L×L) 150×100×100 millimeter.

12. A portable rebreathing system according to claim 9 where said common housing (X) contains a manually adjustable control member (6ar) capable of adjusting the position of the ambient control valve (6a) connected to an ambient air port (7), opening flow to and from the ambient air port to said inhale passage dependent on the position of the manually adjustable control member (6ar).

13. A portable rebreathing system according to claim 10 where the position of said rebreathe control valve (6r) is depending on the oxygen pressure established in the oxygen supply port (5) applied at one side of the rebreathe control valve (6r) body and a counter force from a return spring (6r s) applied on an opposite side of the rebreathe control valve body.

14. A portable rebreathing system according to claim 1 wherein the carbon dioxide scrubber (3/31) is connected with a detachable scrubber connector (3a) to the common valve housing (X) in one end of the carbon dioxide scrubber and in the other end is the carbon dioxide scrubber connected with a detachable counter lung connector (2a) to the counter lung (2), forming a part of the exhale passage from the rebreathe control valve (6r) via said carbon dioxide scrubber and finally to the counter lung (2), said carbon dioxide scrubber also including a by-passing channel (32) connecting the counter lung to the inhale passage.

15. A portable rebreathing system according to claim 1 wherein active chemicals of the carbon dioxide scrubber (31) are contained in a detachable cassette insert able into the housing (3) of the carbon dioxide scrubber when a connector has been detached from the housing (3) of the carbon dioxide scrubber or detached from the common valve housing (X).
